(19) United States
(12) Patent Application Publication
(10) Pub. No.: US 2007/0166722 A1
(43) Pub. Date: Jul. 19, 2007

(54) Detection mycobacterium

(75) Inventor: Jay L.E. Ellingson, Marshfield, WI (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Marshfield Clinic, Marshfield, WI (US)

(21) Appl. No.: 11/200,885

(22) Filed: Aug. 9, 2005

Related U.S. Application Data
(60) Provisional application No. 60/600,148, filed on Aug. 9, 2004.
(60) Provisional application No. 60/600,475, filed on Aug. 10, 2004.

(51) Int. Cl. C12Q 1/68; C12P 19/34

(52) U.S. Cl. 435/6; 435/91.2

(57) ABSTRACT

The present invention provides a method for determining the presence of Mycobacterium avium subspecies paratuberculosis (MAP) in a sample. The method involves using a pair of oligonucleotide probes and detecting the presence or absence of fluorescence resonance energy transfer (FRET) between the donor fluorescent moiety of the first probe and the corresponding acceptor fluorescent moiety of the second probe. The present invention also provides a method for isolating and/or extracting DNA from a microorganism.

### CROSS-REFERENCE TO RELATED APPLICATIONS

This application claims the priority benefit of U.S. Provisional Application Nos. 60/600,148, filed Aug. 9, 2004, and 60/600,475, filed Aug. 10, 2004, which are incorporated herein by reference in their entirety.

### FIELD OF THE INVENTION

The present invention provides a method for detecting the presence of microorganism in a sample.

### BACKGROUND OF THE INVENTION

Microorganisms in a subject or consumables, such as dairy products or meat, often escape detection. Unchecked and undetected, many of these microorganisms cause severe problems to the host as well as spread to the same or other species such as humans. For example, Mycobacterium avium subspecies paratuberculosis (MAP) can be spread from cows to humans through dairy product consumption by humans, and Salmonella can be spread from chicken to humans through egg, egg-product or poultry consumption by humans. Conventional microorganism detection techniques are expensive, slow and/or time consuming often taking days for the results.

Moreover, conventional microorganism detection techniques often require the microorganism to be cultured to a concentration of at least 10⁵/mL to be detected. Because the margin of error in detectability of the microorganism is high, false negative tests may result.

Therefore, there is a need for a test method for rapidly detecting the presence of a microorganism with a high degree of accuracy and reproducibility. There is also a need for testing for the presence of a microorganism in a dilute sample.

### SUMMARY OF THE INVENTION

One aspect of the present invention provides a method for detecting the presence of Mycobacterium avium subspecies paratuberculosis (MAP) in a sample comprising:
amplifying the sample with a pair of hspX gene primers to produce an hspX gene amplification product that comprises the nucleotide sequences of a pair of hspX gene probes if the nucleic acid sequence of MAP hspX gene is present in the sample;
contacting the amplification product with the pair of hspX gene probes, wherein the members of the pair of hspX gene probes hybridize to the amplification product within no more than five nucleotides of each other, wherein a first hspX gene probe of the pair of hspX gene probes is labeled with a donor fluorescent moiety and wherein a second hspX gene probe of the pair of hspX gene probes is labeled with a corresponding acceptor fluorescent moiety; and
detecting the presence or absence of fluorescence resonance energy transfer (FRET) between the donor fluorescent moiety of the first hspX gene probe and the acceptor fluorescent moiety of the second hspX gene probe, wherein the presence of FRET is indicative of the presence of MAP in the sample,
   wherein one of the hspX gene probes comprises no more than 30 nucleotides in length and comprises the sequences 5′-GCA CCC GTC GTG GTA TCT-3′ (SEQ ID NO: 1).

In one particular embodiment, the other hspX gene probe comprises no more than 30 nucleotides in length and comprises the sequences 5′-AAT CTG CAA GCC AAT CCG G-3′ (SEQ ID NO: 2).

In another embodiment, one of the hspX gene primers comprises no more than 30 nucleotides in length and comprises the sequences 5′-GAC CGG CTA TCT GTGOGAA C-3′ (SEQ ID NO:3).

Yet in another embodiment, the other hspX gene primer comprises no more than 30 nucleotides in length and comprises the sequences 5′-CTC GTC GGC TTG CAC CTG-3′ (SEQ ID NO: 4).

Still another aspect of the present invention comprises a method for detecting the presence of Mycobacterium avium subspecies paratuberculosis (MAP) in a sample comprising:
amplifying the sample with a pair of hspX gene primers to produce an hspX gene amplification product that comprises the nucleotide sequences of a pair of hspX gene probes if the nucleic acid sequence of MAP hspX gene is present in the sample;
contacting the amplification product with the pair of hspX gene probes, wherein the members of the pair of hspX gene probes hybridize to the amplification product within no more than five nucleotides of each other, wherein a first hspX gene probe of the pair of hspX gene probes is labeled with a donor fluorescent moiety and wherein a second hspX gene probe of the pair of hspX gene probes is labeled with a corresponding acceptor fluorescent moiety; and
detecting the presence or absence of fluorescence resonance energy transfer (FRET) between the donor fluorescent moiety of the first hspX gene probe and the acceptor fluorescent moiety of the second hspX gene probe, wherein the presence of FRET is indicative of the presence of MAP in the sample,
   wherein one of the hspX gene probes comprises no more than 30 nucleotides in length and comprises the sequences 5′-AAT CTG CAA GCC AAT CCG G-3′ (SEQ ID NO: 2).

In some aspects of the present invention, the donor fluorescent moiety is fluorescein. In such embodiments, the corresponding acceptor fluorescent moiety is preferably LightCycler Red fluorophore.

Typically, the first probe is labeled with the donor fluorescent moiety on the 3′-end and the second probe is labeled with the corresponding acceptor fluorescent moiety on the 5′-end. Often the second probe further comprises a phosphate moiety on the 3′-end.

In some aspects of the present invention, the first hspX gene primer comprises no more than 30 nucleotides in length and comprises the sequences:

| | |
|---|---|
| 5′-GAC CGG CTA TCT GTG GAA C-3′. | (SEQ ID NO:3) |

In other aspects of the present invention, the second hspX gene primer comprises no more than 30 nucleotides in length and comprises the sequences:

| | |
|---|---|
| 5′-CTC GTC GGC TTG CAC CTG-3′. | (SEQ ID NO:4) |

Still another aspect of the present invention provides a test kit for detecting the presence of MAP in a sample. Typically, the test kit is a polymerase chain reaction kit comprising primers and probes described herein.

### DETAILED DESCRIPTION OF THE INVENTION

Definitions

As used herein, the term “subject” means animals, such as mammals, birds, fish, reptiles, etc. Suitable subjects of the present invention include, but are not limited to, human, domesticated mammals of the genus Bos (including cows, steers, bulls, and oxen), chicken, fish, as well as other animals that are raised for meat and/or dairy products.

The term “solid support” means any solid object that is relatively stable and can be used to covalently link an antibody or other suitable material that can be used to form a complex with a microorganism. Exemplary a matrix-filled column, microtiter plate, magnetic beads, glass, silicon, water, etc.

As used herein, the expression “sample” means any clinical, laboratory, environmental or other collected sample of material that is being tested for a microorganism. Exemplary samples include swabs, scrapings or collections of food, bacteriologic cultures, body fluids, tissues, soil, animal feed, or other sources of MAP infection or contamination.

The term “probe” is used herein in the broadest sense to refer to either a labeled or an unlabeled, single-stranded nucleic acid that will hybridize under predetermined conditions of stringency to the target nucleic acid. Such probes may be DNA or RNA and will typically be at least about 10, preferably at least about 15 bases in length, and more preferably about 20-100 bases in length. When used in a hybridization assay, hybrids formed from the probes and the target sequence are usually detected by means of a detectable label affixed directly to the probe. Alternatively, probes can be used as helper probes to facilitate binding of a separate labeled probe to the target nucleotide. It is understood that for hybridization to occur, the probe may or may not be exactly complementary to the target sequence, provided that the hybridization conditions are appropriately selected to permit hybridization even when there are a limited number of mismatches between the respective sequences.

The term “primer” is used herein in its usual sense to be descriptive of an oligonucleotide (DNA or RNA), usually about 10-30 nucleotides in length, and preferably about 12-25 bases in length, that will participate in a primer extension reaction when catalyzed by a polymerase. These reactions are more commonly referred to as “polymerase chain reactions” (“PCR”). Contemplated herein as primers are only those nucleotides that are properly oriented so as to amplify a region within the target sequence.

It should be understood that whenever a nucleotide sequence is given, the scope of the present invention also encompasses the complementary nucleotide sequence.

Unless otherwise indicated, the term “species-specific” is used herein to indicate specificity for a particular subspecies of the microorganism.

The expression “sequence-specific oligonucleotide” is used herein to refer to probes or primers having a hybridizing region that is exactly complementary to a segment of the target region.

General Overview

The present invention provides a method for detecting the presence of MAP in a sample. In particular, methods of the present invention are based on amplification of at least a portion of the genetic material of MAP, preferably the hspX gene of MAP.

One aspect of the invention provides for a method of detecting the presence of Mycobacterium avium subspecies paratuberculosis (MAP) in a sample. Primers and probes for detecting MAP are provided in the present invention, as are kits containing such primers and probes. Methods of the present invention can be used to rapidly detect the presence of MAP DNA from the sample. In one particular embodiment, primers and probes of the present invention are used to amplify and monitor or detect the development of specific amplification products using fluorescence resonance energy transfer (FRET).

The method to detect MAP typically includes performing at least one cycling step, which includes an amplifying step and a hybridizing step. The amplifying step includes amplifying a portion of a MAP hspX gene nucleic acid sequence from the sample using a pair of hspX gene primers, thereby producing an hspX gene amplification product. The hybridizing step includes annealing a pair of hspX gene probes to the hspX gene amplification product. Generally, the members of the pair of hspX gene probes hybridize within no more than five, preferably four, more preferably three, and most preferably one, nucleotide of each other. A first hspX gene probe of the pair of hspX gene probes is typically labeled with a donor fluorescent moiety and a second hspX gene probe of the pair of hspX gene probes is labeled with a corresponding acceptor fluorescent moiety.

The method further includes detecting the presence or absence of FRET between the donor fluorescent moiety of the first hspX gene probe and the acceptor fluorescent moiety of the second hspX gene probe upon hybridization of the pair of hspX gene probes to the amplification product. The presence of FRET is usually indicative of the presence of MAP in the sample, while the absence of FRET is usually indicative of the absence of MAP in the sample.

In one aspect, the detecting step includes exciting the sample and/or the amplification product at a wavelength absorbed by the donor fluorescent moiety and visualizing and/or measuring the wavelength emitted by the acceptor fluorescent moiety (i.e., visualizing and/or measuring FRET). In another aspect, the detecting step includes quantitating the FRET. In yet another aspect, the detecting step can be performed after each cycling step (e.g., in real-time).

Generally, the presence of FRET within 50 cycles (e.g., 20, 25, 30, 35, 40, or 45 cycles) indicates the presence of a MAP in the sample. In addition, determining the melting temperature between one or both of the hspX gene probe(s) and the hspX gene amplification product can confirm the presence or absence of MAP.

The cycling step can be performed on a control sample. A control sample can include the same portion of the MAP hspX gene nucleotide. Alternatively, a control sample can include a nucleic acid molecule other than MAP hspX gene nucleotide. Cycling steps can be performed on such a control sample using a pair of control primers and a pair of control probes. The control primers and probes can be other than hspX gene primers and probes. One or more amplifying steps produces a control amplification product. Each of the control probes hybridizes to the control amplification product.

In another aspect of the invention, there are provided articles of manufacture, or kits for testing the presence of MAP in a sample. Articles of manufacture can include fluorophoric moieties for labeling the probes or probes already labeled with donor and corresponding acceptor fluorescent moieties. The article of manufacture can also include a package insert having instructions thereon for using the primers, probes, and fluorophoric moieties to detect the presence or absence of MAP in a sample.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

In another aspect, methods of the present invention are useful in detecting MAP that is present in a minute quantity. For example, methods of the present invention allow detection of microorganism that is present in a sample at a concentration of about 10 microorganism per 5 g of sample or lower, and preferably 1 microorganism per 5 g of sample.

In another embodiment, methods of the present invention provide rapid and MAP-specific detection in a sample. Such methods include amplifying at least a portion of the genetic material of MAP, preferably hspX gene, in the presence of at least one appropriate detection material, such as a probe. In this manner, methods of the present invention are able to detect the presence of MAP in a sample in less than 2 days, preferably in less than 1 day, and more preferably in less than a 20 hour period.

One particular aspect of the present invention provides a real-time assay for detecting MAP in a sample that is more sensitive than conventional assays. Primers and probes for detecting MAP and articles of manufacture containing such primers and probes are provided. The increased sensitivity of real-time PCR for detection of MAP compared to other methods, as well as the improved features of real-time PCR including sample containment and real-time detection of the amplified product, make feasible the implementation of this technology for routine diagnosis of MAP detection in the clinical laboratory.

Samples For Testing

Methods of the present invention can be used to test a wide variety of materials for the presence of MAP. Generally, any material that is believed to contain MAP is suitable for testing. For commercial purposes, however, methods of the present invention are typically used to detect the presence of MAP in food products, soil, and subject's body fluids. Exemplary materials that are suitable samples for testing include, but are not limited to, food products (such as raw meat, meat by-products (e.g., hot dogs, sausages, etc.), milk and milk by-products, such as cheese and butter, as well as any consumable food products), subject's body fluids (such as, blood, intestinal fluid, saliva, urine, stool, lymph fluid, spinal fluid, tears, nasal secretions, and other subject's excrements), and subject's tissue samples.

Mycobacterium avium subspecies paratuberculosis (MAP)

Conventional commercial MAP detection methods involve an enzymelinked immunosorbent assay (i.e., ELISA), where a specific antibody is linked to a solid substrate such as a microtiter plate. The MAP's antigen is recognized by the antibody and forms an antigen-antibody complex. Detection is achieved by reacting a second antigen-specific antibody linked to an enzyme with the bound protein. A substrate solution is then applied that results in a colored substrate when catalyzed by the linked enzyme. These results are then read by an individual or an ELISA reading instrument. This assay is expensive, slow and time consuming, often taking days for the results.

While ELISA is based on the analysis of an antigen (i.e., a protein), methods of the present invention are based in part on the analysis of MAP's genetic material. The presence of MAP can be determined by analyzing the sample for the presence of hspX gene of MAP. HspX gene distinguishes the presence of MAP from other microorganisms such as other mycobacterium.

HspX gene is present as a single-copy gene in the MAP genome. This gene provides a unique target region for the construction of suitable probes and primers that are species-specific for distinguishing MAP from related mycobacteria in a test sample. One embodiment of the present invention provides a test kit detecting the presence of MAP in a sample by analyzing the presence or absence of hspX gene within the sample.

MAP nucleic acids other than those exemplified herein (e.g., other than hspX gene, such as IS900) also can be used to detect MAP in a sample and are known to those of skill in the art. The nucleic acid sequence of the MAP genome, as well as MAP hspX gene are available. See, for example, U.S. Pat. Nos. 5,985,576 and 6,277,580 which are incorporated herein by reference in their entirety. Specifically, primers and probes to amplify and detect MAP hspX gene nucleic acid molecules are provided by the present invention.

Primers that amplify hspX gene of MAP can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 10 to 30 nucleotides in length.

Designing oligonucleotides to be used as hybridization probes can be performed in a manner similar to the design of primers, although the members of a pair of probes preferably anneal to an amplification product within no more than 6 nucleotides of each other on the same strand such that FRET can occur (e.g., within no more than 1, 2, 3, 4 or 5 nucleotides of each other). This minimal degree of separation typically brings the respective fluorescent moieties into sufficient proximity such that FRET occurs. It is to be understood, however, that other separation distances (e.g., 7 or more nucleotides) are possible provided the fluorescent moieties are appropriately positioned relative to each other (for example, with a linker arm) such that FRET can occur. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 10 to 30 nucleotides in length.

Amplification

Methods of the present invention include providing conditions that allow amplification of at least a portion of MAP's genetic material. Preferably, amplification conditions allow a species-specific amplification. However, it should be appreciated that amplification conditions of the present invention need not be 100% species-specific. As long as the amplification results in the detection accuracy rate of at least 95%, preferably at least 98% and more preferably at least 99%, it is well within the scope of the present invention.

While the scope of the present invention includes any method (for example, Polymerase Chain Reaction, i.e., PCR, and nucleic acid sequence based amplification, i.e., NASBA) for amplifying at least a portion of MAP's genetic material, for brevity sake, the present invention will now be described in reference to PCR technique.

Amplification of a genetic material, e.g., DNA, is well known in the art. See, for example, U.S. Pat. Nos. 4,683,202, 4,683,195, 4,800,159, 4,965,188, and 4,994,370, which are incorporated herein by reference in their entirety. Methods of the present invention include providing conditions that would allow amplification of the microorganism's genetic material, if the microorganism is present in the sample. In this manner, detection of the amplification product is indicative of the presence of the microorganism in the sample.

By knowing the nucleotide sequences of the genetic material in the microorganism of interest, one can design a specific primer sequence. Typically, the primer is about 5 to 30 oligonucleotides long, and preferably about 10 to 20 oligonucleotides. This primer length is only an illustrative example, and the present invention is not limited to this particular primer sequence length. Once the suitable primer sequences are selected, they can readily be synthesized or can be obtained from third parties, such as Roche Diagnostics, etc. Other reagents, such as DNA polymerases and nucleotides, that are necessary for a PCR amplification are also commercially available.

Fluorescence Resonance Energy Transfer (FRET)

FRET technology (see, for example, U.S. Pat. Nos. 4,996,143, 5,565,322, 5,849,489, and 6,162,603) is based on a concept that when a donor and a corresponding acceptor fluorescent moiety are positioned within a certain distance of each other, energy transfer takes place between the two fluorescent moieties that can be visualized or otherwise detected and/or quantitated. As used herein, two oligonucleotide probes, each containing a fluorescent moiety, can hybridize to an amplification product at particular positions determined by the complementarity of the oligonucleotide probes to the MAP target nucleic acid sequence. Upon hybridization of the oligonucleotide probes to the amplification product nucleic acid at the appropriate positions, a FRET signal is generated. 100481 Fluorescent analysis can be carried out using, for example, a photon counting epifluorescent microscope system (containing the appropriate dichroic mirror and filters for monitoring fluorescent emission at the particular range), a photon counting photomultiplier system or a fluorometer. Excitation to initiate energy transfer can be carried out with an argon ion laser, a high intensity mercury (Hg) arc lamp, a fiber optic light source, or other high intensity light source appropriately filtered for excitation in the desired range.

As used herein with respect to donor and corresponding acceptor fluorescent moieties “corresponding” refers to an acceptor fluorescent moiety having an emission spectrum that overlaps the excitation spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced therebetween.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinirndyl 1-pyrenebutyrate, and 4-acetamido-4′-isothiocyanatostilbene-2,2′-disulfonic acid derivatives.

Representative acceptor fluorescent moieties, depending upon the donor fluorescent moiety used, include LC-Red 640, LC-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm for the purpose of the present invention is the distance in Angstroms (Å) from the nucleotide base to the fluorescent moiety. In general, a linker arm is from about 10 to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety such as an LC-Red 640-NHS-ester can be combined with C₆-Phosphoramidites (available from ABI (Foster City, Calif.) or Glen Research (Sterling, Va.)) to produce, for example, LC-Red 640-Phosphoramidite. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as fluorescein-CPG from BioGenex (San Ramon, Calif.)), or 3′-amino-CPG's that require coupling of a fluorescein-NHS-ester after oligonucleotide synthesis.

Detection

By using commercially available real-time PCR instrumentation (e.g., LightCycler®, Roche Molecular Biochemicals, Indianapolis, Ind.), PCR amplification and detection of the amplification product can be combined in a single closed cuvette with dramatically reduced cycling time. Since detection occurs concurrently with amplification, the real-time PCR methods obviate the need for manipulation of the amplification product, and diminish the risk of cross-contamination between amplification products. Real-time PCR greatly reduces turn-around time and is an attractive alternative to conventional PCR techniques in the clinical laboratory.

The present invention provides methods for detecting the presence or absence of MAP in a sample. Methods provided by the invention avoid problems of sample contamination, false negatives, and false positives. The methods include performing at least one cycling step that includes amplifying the hspX gene of MAP from a sample using a pair of hspX gene primers. Each of the hspX primers anneals to a target within or adjacent to the hspX gene such that at least a portion of the amplification product contains nucleic acid sequence corresponding to the hspX gene and, more importantly, such that the amplification product contains the nucleic acid sequences that are complementary to the hspX gene probes. The hspX gene amplification product is produced provided that MAP nucleic acid is present. Each cycling step further includes hybridizing a pair of hspX gene probes to the hspX gene amplification product. According to the invention, one of the hspX gene probes is labeled with a donor fluorescent moiety and the other is labeled with a corresponding acceptor fluorescent moiety. The presence or absence of FRET between the donor fluorescent moiety of the first hspX gene probe and the corresponding acceptor fluorescent moiety of the second hspX gene probe is detected upon hybridization of both hspX gene probes to the hspX gene amplification product.

Each cycling step includes an amplification step and a hybridization step, and each cycling step is usually followed by a FRET detecting step. Multiple cycling steps are performed, preferably in a thermocycler. The above-described methods for detecting MAP in a sample using primers and probes directed toward hspX gene also can be performed using other MAP gene-specific primers and probes.

As used herein, “amplifying” refers to the process of synthesizing nucleic acid molecules that are complementary to one or both strands of a template nucleic acid molecule (e.g., hspX gene). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleotide triphosphates, a DNA polymerase enzyme (e.g., Platinum Taq) and an appropriate buffer and/or co-factors for optimal activity of the polymerase enzyme (e.g., MgCl₂ and/or KCl).

If amplification of hspX gene occurs and an amplification product is produced, the step of hybridizing results in a detectable signal based upon FRET between the pair of probes. As used herein, “hybridizing” refers to the annealing of probes to an amplification product. Hybridization conditions typically include a temperature that is below the melting temperature of the probes but that avoids non-specific hybridization of the probes.

Generally, the presence of FRET indicates the presence of MAP in the sample, and the absence of FRET indicates the absence of MAP in the sample. Inadequate specimen collection, transportation delays, inappropriate transportation conditions, or use of certain collection swabs (calcium alginate or aluminum shaft) are all conditions that can affect the success and/or accuracy of a test result, however.

Using the methods disclosed herein, detection of FRET within 30 cycling steps is indicative of the presence of MAP. Samples in which FRET is detected after more than 30 cycling steps also is indicative of the presence of MAP. The cycle number at which FRET is detectable can be correlated with the amount of MAP in a sample.

The presence or absence of PCR amplification product can be detected by any of the techniques known to one skilled in the art. In one particular embodiment, methods of the present invention include detecting the presence or absence of the PCR amplification product using a probe that hybridizes to a particular genetic material of the microorganism. By designing the PCR primer sequence and the probe nucleotide sequence to hybridize different portions of the microorganism's genetic material, one can increase the accuracy and/or sensitivity of the methods disclosed herein.

While there are a variety of labeled probes that are available, such as radio-active and fluorescent labeled probes, as described herein in one particular embodiment, methods of the present invention use a fluorescence resonance energy transfer labeled probe as internal hybridization probes. In one particular embodiment of the present invention, an internal hybridization probe is included in the PCR reaction mixture so that product detection occurs as the PCR amplification product is formed, thereby reducing post-PCR processing time. Roche Lightcycler PCR instrument (U.S. Pat. No. 6,174,670) or other real-time PCR instruments can be used in this embodiment of the invention, e.g., see U.S. Pat. No. 6,814,934. PCR amplification of a genetic material increases the sensitivity of methods of the present invention to 10³, preferably 10² and more preferably 10¹, microorganisms or less in comparison to about 10⁵ microorganisms that are required in standard ELISA methods. In some instances, real-time PCR amplification and detection significantly reduce the total assay time so that test results may be obtained in less than or within 12 hours. Accordingly, methods of the present invention provide rapid and/or highly accurate results relative to the conventional methods.

In one particular embodiment of the present invention, methods for determining the presence of MAP utilize PCR amplification using a least one of the following primers, preferably both:

forward:

(SEQ ID NO:7)

5′-(CGA) GAC CGG CTA TCT GTG GAA C (GGC)-3′

and

reverse:

(SEQ ID NO:8)

5′-(CCA) CTC GTC GGC TTG CAC CTG (AAT)-3′

Throughout herein, each of the nucleotide sequences within the parenthesis is optional, i.e., it can be independently absent or present. Use of SEQ ID NO:7 and SEQ ID NO:8 as primers, including the sequences in the parenthesis, provides a 209 bp product spanning from base 191 to base 399 of the hspX gene of the MAP genome.

In addition, methods for determining the presence of MAP can also include one or two, preferably both, hybridization probes that are designed to allow for detection of the PCR product by Fluorescence Resonance Energy Transfer (FRET), for example, within the Lightcycler (Roche). The sequence and modifications of the probes are:
upstream: 5′-(GCG) GCA CCC GTC GTG GTA TCT (G)-Fluorescein
downstream: 5′-LC Red-(G)AA TCT GCA AGC CAA TCC GG(C GG)-Phosphorylation-3′.

These probes anneal to the upper strand from positions 228-246 (upstream) and 247-266 downstream. Each of the nucleotides within the parenthesis is optional; however, inclusion of these nucleotide sequences increases the species-specificity. Use of this primer and probe combination in a real time PCR system greatly improve sensitivity, specificity, and turn around time for the detection of MAP, for example, in dairy products, fecal samples, biological samples and enviromnental monitoring.

Isolation of DNA

Some embodiments of the present invention also include a process for isolating MAP DNA from a very dilute sample. This is particularly useful in situations where the total number of microorganisms present in the sample is sufficient for analysis but the volume of sample is simply too large. Accordingly, one aspect of the present invention provides a process of isolating MAP from a dilute sample without a need to increase the total amount of MAP in the sample, e.g., via culturing.

Suitable conditions for culturing a variety of microorganisms are well known to one skilled in the art. This is especially true for food-borne pathogenic bacteria, viruses, and other pathogenic microorganisms. It should be appreciated that if the sample does not contain the microorganism to be detected, subjecting the sample to a suitable culturing conditions for the microorganism will not result in any increase in its number. In this case, a subsequent analysis by methods described above, i.e., PCR amplification and detection, will result in no detection of any PCR amplification product. This is an indication that no microorganism of interest was present in the sample. While culturing the sample to increase the total amount of MAP for easy detection is well known in the art, it increase the total analysis time. Another aspect of the present invention eliminates the need to culture MAP in the sample by providing conditions that allow isolation of sufficient MAP DNA in a very dilute sample (i.e., <10⁵ MAP per sample, preferably 10³, preferably 10² and more preferably 10¹, MAP per sample)

Whether the sample is subjected to appropriate microorganism culturing conditions or not, in order to analyze a dilute sample for the presence of a microorganism, some embodiments of the present invention include placing the sample in an aqueous solution and centrifuging the resulting aqueous solution for about 15 min. The speed of centrifugation of the aqueous solution is typically at least about 3000×g force, preferably at least about 3,900×g force. Often the sample is centrifuged from about 4,000 to about 6,000×g force for about 10 to 20 minutes, generally about 15 minutes.

The supernatant is discarded and the resulting sample (e.g., pellet) is resuspended in a phosphate buffer solution (PBS) and a positively charged beads, e.g., zirconia/silica beads as well as other positively charged beads that are well known to one skilled in the art, is added to the mixture. The mixture containing the beads is then centrifuged for about 3 to 6 minutes, typically for about 3 minutes. The speed of centrifugation is typically at least about 9,500×g force, preferably at least about 10,000×g force. Generally, the mixture is centrifuged at from about 10,000 to about 12,000×g force. The supernatant is again removed, preferably without disturbing the pellet and beads.

Cell lysis buffer solution (i.e., a solution for nucleic acid isolation) is then added to the pellet and beads. Suitable lysis buffer solutions are well known to one skilled in the art and are commercially readily available, for example, from Roche Applied Science (such as MagNA Pure LC System). The resulting mixture is typically incubated at about 95° C. for about 10 minutes, cooled to room temperature, centrifuged at about 65 rpm for about 45 seconds, typically using the MagNA Lyser instrument (Roche Applied Science). The resulting sample is then centrifuged at the rate of at least about 9,500×g force, preferably at least about 10,000×g forceand more preferably at about 10,000 to about 12,000×g force, for about 3 to 6 minutes, typically about 3 minutes in a benchtop centrifuge.

The resulting supernatant is then separated and DNA material is extracted, for example, using commercially available DNA extraction kit such as MagNA Pure automated DNA extraction instrument and the MagNA Pure extraction kit III. The extracted DNA material is further centrifuged at a rate of at least about 9,500×g force, preferably at least about 10,000×g force, and more preferably at about 10,000 to about 12,000×g force.

Other Microorganisms

Methods of the present invention are useful in detecting the presence of any microorganism that has a genetic material, such as, DNA, RNA or mitochondria nucleic acids. Accordingly, the presence of a wide variety of microorganisms, such as bacteria and viruses, can be detected using methods of the present invention.

In one particular embodiment, methods of the present invention are used to detect the presence of food-borne pathogenic bacteria, viruses, and other pathogenic microorganisms. Exemplary microorganisms that can be detected by methods of the present invention include, but not limited to, Mycobacterium avium subspecies paratuberculosis (MAP), B. anthracis (i.e., anthrax), Listeria, Salmonella, E. Coli (such as EHEC), viruses (e.g., norwalk), as well as, waterborne microorganisms, such as Cryptosporidium, and viruses and bacteria that cause meningitis.

### EXAMPLES

### Example 1

This example illustrates a method for extracting MAP from a dilute sample.

About 4 g of fecal material is placed into a sterile 50 mL plastic conical tube containing 35 ml of sterile water. The resulting mixture is shaken vigorously for 15 seconds and the tube is allowed to sit upright at room temp for about 30 minutes. With a sterile disposable pipette, about 15 mL of liquid from the top portion of the tube it transferred into a 15 mL tube. The resulting mixture is centrifuged at 3,900 times the gravitational force (i.e., “×g”) for 15 minutes. The supernatant is discarded and the resulting residue (i.e., pellet) is resuspend in 500 μL of PBS. The resuspended pellet is transferred to a 1.5 mL sterile microcentrifuge tube containing zirconia/silica beads and is centrifuged at 10,000×g for 3 minutes in a benchtop centrifuge.

The supernatant is removed and discarded without disturbing the pellet and beads. About 300 μL of lysis buffer (from MagNA Pure LC DNA Isolation Kit III (bacterial, fungi) is added to each tube and incubate at 95° C. for 10 minutes. The samples are cooled to room temperature and then centrifuged for 45 seconds at 65 rpm in the MagNA Lyser instrument followed by centrifuging at 10,000×g for 3 minutes in a benchtop centrifuge.

About 250 μL of supernatant is removed and extracted using the Magna Pure automated DNA extraction instrument and the Magna Pure extraction kit III (bacteria, fungi) according to the manufacturers instructions (Roche Applied Science). The extracted DNA is transferred to a 1.5 mL sterile microcentrifuge tube and centrifuged at 10,000×g for 3 minutes in a benchtop centrifuge. The supernatant is separated to obtain the extracted DNA.

### Example 2

This example illustrates PCR amplification using P90/P91 PCR primers.

The extracted DNA in the supernatant in Example 1 above is amplified using P90/P91 PCR primers on the Lightcycler:

| | |
|---|---|
| Sequence P90- | |
| 5′-GAA GGG TGT TCG GGG CCG TCG CTT | (SEQ ID NO:5) |
| AGG-3′ | |
| Sequence P91- | |
| 5′-GGC GTT GAG GTC GAT CGC CCA CGT | (SEQ ID NO:6) |
| GAC-3′ | |

The PCR mixture contained the following chemicals: 12.8 μL of PCR-grade water, 1.2 μL of 25 mM aqueous solution of MgCl₂, 2.0 μL of DNA Master, 1.0 μL of P90(forward primer), 1.0 μL of P91 (reverse primer) for a total of 18.0 μL. The primers are at concentration of 20 pmol/μL.

The amplification reactions containing 18 μL of master mix and 2 μL extracted DNA template solution (Example 1) was added to each capillary. Load capillaries into carousel and centrifuge in LC centrifuge. Amplification conditions are as follows: 10 min at 95° C.; 40 cycles of 10 sec at 95° C., 5 sec at 75° C., and 16 sec at 72° C. with a single fluorescence acquisition during each cycle. Melting curve conditions are as follows: 0 sec at 95° C.; 1 min at 70° C.; and 0 sec at 99° C. with a 0.1 C/sec slope and continuous acquisition. Cooling cycle was 30 sec at 40° C. The PCR is monitored by using the double-stranded DNA binding dye SYBER Green (Applied Biosystems).

A sample is considered positive for the presence of MAP if typical colonies collected during the slant rinsing procedure are PCR positive with the P90/P91 primer showing an increase in fluorescence during amplification and the corresponding melting curve can be observed at 90-92° C.

### Example 3

This example illustrates PCR amplification of a portion of the hspX gene.

The extracted DNA in the supernatant in Example 1 above is amplified using hspX PCR primers on the Lightcycler:

| | |
|---|---|
| 5′-GAC CGG CTA TCT GTG GAA C-3′ | (SEQ ID NO:3) |
| 5′-CTC GTC GGC TTG CAC CTG-3′ | (SEQ ID NO:4) |

The PCR mixture contained the following chemicals: 9.0 μL of PCR-grade water, 2.0 μL of 25 mM aqueous MgCl₂ solution, 2.0 μL of DNA Master, 1.0 μL of SEQ ID NO:3 (forward primer)*, 1.0 μL of SEQ ID NO:4 (reverse primer)*, 1.0 μL of upstream probe (SEQ ID NO:1) and 2.0 μL of downstream probe (SEQ ID NO: 2) for a total of 18.0 μL. The primers are at concentration of 20 pmol/μL, probes are at concentration of 4 pmol/μL.

The amplification reactions containing 18 μL of master mix and 2 μL of extracted DNA template solution (Example 1) are added to each capillary. Load capillaries into carousel and centrifuge in LC centrifuige. Amplification conditions are as follows: 10 min at 95° C.; 40 cycles of 10 sec at 95° C., 5 sec at 55° C., and 10 sec at 72° C. with a single fluorescence acquisition during each cycle. Melting curve conditions are as follows: 0 sec at 95° C.; 1 min at 35° C.; and 0 sec at 85° C. with a 0.1° C./sec slope and continuous acquisition. Cooling cycle was 30 sec at 40° C. The PCR is monitored by FRET between upstream and downstream probes.

A sample is considered positive for the presence of MAP if: (1) extracted DNA template solution (Example 1) is PCR positive with the hspX primers showing an increase in fluorescence during amplification and the corresponding melting curve can be observed at 60-63° C.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for detecting the presence of Mycobacterium avium subspecies paratuberculosis (MAP) in a sample comprising:
amplifying the sample with a pair of hspX gene primers to produce an hspX gene amplification product that comprises the nucleotide sequences of a pair of hspX gene probes if the nucleic acid sequence of MAP hspX gene is present in the sample;
contacting the amplification product with the pair of hspX gene probes, wherein the members of the pair of hspX gene probes hybridize to the amplification product within no more than five nucleotides of each other, wherein a first hspX gene probe of the pair of hspX gene probes is labeled with a donor fluorescent moiety and wherein a second hspX gene probe of the pair of hspX gene probes is labeled with a corresponding acceptor fluorescent moiety; and
detecting the presence or absence of fluorescence resonance energy transfer (FRET) between the donor fluorescent moiety of the first hspX gene probe and the acceptor fluorescent moiety of the second hspX gene probe, wherein the presence of FRET is indicative of the presence of MAP in the sample,
wherein one of the hspX gene probes comprises no more than 30 nucleotides in length and comprises the sequence 5′-GCA CCC GTC GTG GTA TCT-3′ (SEQ ID NO:1).

2. The method of claim 1, wherein the other hspX gene probe comprises no more than 30 nucleotides in length and comprises the sequences 5′-AAT CTG CAA GCC AAT CCG G-3′ (SEQ ID NO: 2).

3. The method of claim 1, wherein one of the hspX gene primers comprises no more than 30 nucleotides in length and comprises the sequences 5′-GAC CGG CTA TCT GTG GAA C-3′ (SEQ ID NO:3).

4. The method of claim 3, wherein the other hspX gene primer comprises no more than 30 nucleotides in length and comprises the sequences 5′-CTC GTC GGC TTG CAC CTG-3′ (SEQ ID NO: 4).

5. The method of claim 1, wherein both steps of amplifying the sample and contacting the amplified product with the pair of hspX gene probes are conducted in a single reaction vessel.

6. A method for detecting the presence of Mycobacterium avium subspecies paratuberculosis (MAP) in a sample comprising:
amplifying the sample with a pair of hspX gene primers to produce a hspX gene amplification product that comprises the nucleotide sequences of a pair of hspX gene probes if the nucleic acid sequence of MAP hspX gene is present in the sample;
contacting the amplification product with the pair of hspX gene probes, wherein the members of the pair of hspX gene probes hybridize to the amplification product within no more than five nucleotides of each other, wherein a first hspX gene probe of the pair of hspX gene probes is labeled with a donor fluorescent moiety and wherein a second hspX gene probe of the pair of hspX gene probes is labeled with a corresponding acceptor fluorescent moiety; and
detecting the presence or absence of fluorescence resonance energy transfer (FRET) between the donor fluorescent moiety of the first hspX gene probe and the acceptor fluorescent moiety of the second hspX gene probe, wherein the presence of FRET is indicative of the presence of MAP in the sample,
wherein one of the hspX gene probes comprises no more than 30 nucleotides in length and comprises the sequences 5′-AAT CTG CAA GCC AAT CCG G-3′ (SEQ ID NO: 2).

7. A pair of MAP hspX gene probes for detecting the presence of MAP in a sample comprising:
a first probe comprising no more than 30 nucleotides in length and comprises the sequences 5′-GAC CGG CTA TCT GTG GAA C-3′ (SEQ ID NO:3);
a second probe comprising no more than 30 nucleotides in length and comprises the sequences 5′-CTC GTC GGC TTG CAC CTG-3′ (SEQ ID NO: 4);
wherein when the two probes are hybridized to MAP hspX gene 3′-end of the first probe is separated from the 5′-end of the second probe by no more than five nucleotides, and wherein the first hspX gene probe is labeled with a donor or an acceptor fluorescent moiety on the 3′-end and the second hspX gene probe is labeled with the corresponding acceptor or donor fluorescent moiety, respectively, on the 5′-end.

8. The pair of MAP hspX gene probes of claim 7, wherein the donor fluorescent moiety is fluorescein.

9. The pair of MAP hspX gene probes of claim 8, wherein the corresponding acceptor fluorescent moiety is LightCycler Red fluorophore.

10. The pair of MAP hspX gene probes of claim 7, wherein the first probe is labeled with the donor fluorescent moiety on the 3′-end and the second probe is labeled with the corresponding acceptor fluorescent moiety on the 5′-end.

11. The pair of MAP hspX gene probes of claim 10, wherein the second probe further comprises phosphate moiety on the 3′-end.

12. The pair of MAP hspX gene probes of claim 10, wherein the 3′-end of the first probe is labeled with fluorescein and the 5′-end of the second probe is labeled with the corresponding acceptor fluorescent moiety.

13. The pair of MAP hspX gene probes of claim 12, wherein the 5′-end of the second probe is labeled with LightCycler Red fluorophore.

14. A method for detecting the presence of Mycobacterium avium subspecies paratuberculosis (MAP) in a sample comprising:
amplifying the sample with a pair of hspX gene primers in the presence of a pair of hspX gene probes such that the amplification produces a hspX gene amplification product when MAP is present in the sample and the pair of hspX gene probes hybridize to the amplification product within no more than five nucleotides of each other, wherein the 3′-end of a first hspX gene probe of the pair of hspX gene probes is labeled with a donor fluorescent moiety, and wherein the 5′-end of a second hspX gene probe of the pair of hspX gene probes is labeled with a corresponding acceptor fluorescent moiety and its 3′-end comprises a phosphate moiety; and
detecting the presence or absence of fluorescence resonance energy transfer (FRET) between the donor fluorescent moiety of the first hspX gene probe and the corresponding acceptor fluorescent moiety of the second hspX gene probe, wherein the presence of FRET is indicative of the presence of MAP in the sample,
wherein
the first hspX gene primer comprising no more than 30 nucleotides in length and comprises the sequences:

| | |
|---|---|
| 5′-GAC CGG CTA TCT GTG GAA C-3′; | (SEQ ID NO:3) |

the second hspX gene primer comprising no more than 30 nucleotides in length and comprises the sequences:

| | |
|---|---|
| 5′-CTC GTC GGC TTG CAC CTG-3′; | (SEQ ID NO:4) |

the first hspX gene probe comprises no more than 30 nucleotides in length, and comprises the sequences:

| | |
|---|---|
| 5′-GCA CCC GTC GTG GTA TCT-3′; | (SEQ ID NO:1) |
| and | |

the second hspX gene probe comprises no more than 30 nucleotides in length, and comprises the sequences:

| | |
|---|---|
| 5′-AAT CTG CAA GCC AAT CCG G-3′. | (SEQ ID NO:2) |

15. A method for separating a microorganism's DNA material from a sample comprising the microorganism, said method comprising:
centrifuging a solution comprising the sample, a cationic solid material, and a phosphate buffer solution at a rate of at least about 9,500×g to produce a separated supernatant and a solid;
removing the supernatant;
adding a lysis buffer solution to the solid to produce a lysing mixture;
heating the lysing mixture to release the microorganism's DNA material into the solution; and
centrifuging the lysing mixture at a rate of at least about 9,500×g, whereby the resulting supernatant comprises the microorganism's separated DNA material.

16. A kit for detecting Mycobacterium avium subspecies paratuberculosis (MAP) in a sample comprising:
a pair of hspX gene probes, wherein the members of the pair of hspX gene probes are capable of hybridizing to the nucleotide sequences of MAP hspX gene within no more than five nucleotides of each other when MAP hspX gene is present, wherein a first hspX gene probe of the pair of hspX gene probes is labeled with a donor fluorescent moiety and wherein a second hspX gene probe of the pair of hspX gene probes is labeled with a corresponding acceptor fluorescent moiety; and
a pair of hspX gene primers suitable for producing an hspX gene amplification product that comprises the nucleotide sequences of the pair of hspX gene probes if the nucleic acid sequence of MAP hspX gene is present in the sample.

17. The kit of claim 16, wherein the first hspX gene probe comprises no more than 30 nucleotides in length, and comprises the sequences:

| | |
|---|---|
| 5′-GCA CCC GTC GTG GTA TCT-3′. | (SEQ ID NO:1) |

18. The kit of claim 17, wherein the second hspX gene probe comprises no more than 30 nucleotides in length, and comprises the sequences:

| | |
|---|---|
| 5′-AAT CTG CAA GCC AAT CCG G-3′. | (SEQ ID NO:2) |

19. The kit of claim 17, wherein the first hspX gene primer comprises no more than 30 nucleotides in length and comprises the sequences:

| | |
|---|---|
| 5′-GAC CGG CTA TCT GTG GAA C-3′. | (SEQ ID NO:3) |

20. The kit of claim 19, wherein the second hspX gene primer comprises no more than 30 nucleotides in length and comprises the sequences:

| | |
|---|---|
| 5′-CTC GTC GGC TTG CAC CTG-3′. | (SEQ ID NO:4) |


SEQUENCE LISTING (8 sequences)

SEQ ID NO: 1 - DNA, 18 bp, Mycobacterium avium subspecies paratuberculosis
gcacccgtcgtggtatct

SEQ ID NO: 2 - DNA, 19 bp, Mycobacterium avium subspecies paratuberculosis
aatctgcaagccaatccgg

SEQ ID NO: 3 - DNA, 19 bp, Mycobacterium avium subspecies paratuberculosis
gaccggctatctgtggaac

SEQ ID NO: 4 - DNA, 18 bp, Mycobacterium avium subspecies paratuberculosis
ctcgtcggcttgcacctg

SEQ ID NO: 5 - DNA, 27 bp, Mycobacterium avium subspecies paratuberculosis
gaagggtgttcggggccgtcgcttagg

SEQ ID NO: 6 - DNA, 27 bp, Mycobacterium avium subspecies paratuberculosis
ggcgttgaggtcgatcgcccacgtgac

SEQ ID NO: 7 - DNA, 25 bp, Mycobacterium avium subspecies paratuberculosis
cgagaccggctatctgtggaacggc

SEQ ID NO: 8 - DNA, 24 bp, Mycobacterium avium subspecies paratuberculosis
ccactcgtcggcttgcacctgaat